(19) United States
(12) Patent Application Publication
(10) Pub. No.: US 2023/0263574 A1
(43) Pub. Date: Aug. 24, 2023

(54) SYSTEMS AND METHOS FOR MONITORING PROXIMITY BETWEEN ROBOTIC MANIPULATORS

(71) Applicant: Asensus Surgical US, Inc., Durham, NC (US)

(72) Inventors: Kevin Andrew Hufford, Cary, NC (US); Matthew Robert Penny, Holly Springs, NC (US); Tal Nir, Haifa (IL); Anthony Fernando, Chapel Hill, NC (US)

(21) Appl. No.: 18/091,282

(22) Filed: Dec. 29, 2022

Related U.S. Application Data
(60) Provisional application No. 63/294,831, filed on Dec. 29, 2021.

(51) Int. Cl. A61B 34/20

(52) U.S. Cl. CPC A61B 34/20; A61B 2034/2055

(57) ABSTRACT

A proximity detection system allows monitoring of proximity between the end effectors of first and second independent robotic manipulators. Imagers are circumferentially positioned around the end effector of at least one of the robotic manipulators. Image data from the imagers is analyzed to determine proximity between the end effectors. When determined proximity falls below a defined threshold, the system issues an alert to the user or slows/suspends manipulator motion.

### BACKGROUND

In robotic surgery, awareness of the proximity between robotic manipulators and other manipulators, equipment or personnel in the operating room is beneficial for avoiding unintended contact or collisions. For surgical robotic systems having multiple arms that emanate from a common base, monitoring the relative position can be performed simply based on known kinematics. For surgical robotic systems in which the robotic arms are mounted on separate carts that may be individually moved, acquiring the relative positioning is more difficult.

In some robotic surgical systems, a force-torque sensor and/or an IMU (inertial measurement unit)/accelerometer may be used to collect information from the surgical site as well as to detect collisions between the most distal portions of manipulators. However, it may be further desirable to predict or detect collisions between not only the most distal portions of the manipulator, but also more proximal portions that may be on the more proximal side of a distally positioned force-torque sensor.

This application describes systems and methods for monitoring proximity between components of robotic manipulators (or other components or personnel within an operating room) in order to avoid unintentional contact between them.

Commonly owned US Publication No. US/2020/0205911, which is incorporated by reference, describes use of computer vision to determine the relative positions of manipulator bases within the operating room. As described in that application, one or more cameras are positioned to generate images of a portion of the operating room, including the robotic manipulators, or instruments carried by the robotic manipulators. Image processing is used to detect the robotic system components on the images captured by the camera. Once the components are detected in the image for each manipulator, the relative positions of the bases within the room may be determined. Concepts described in that application are relevant to the present disclosure, and may be combined with the features or steps disclosed in this application.

Commonly owned and co-pending application Ser. No. 17/944,170, filed Sep. 13, 2022, which is incorporated herein by reference, also describes concepts that may be combined with the features or steps disclosed in this application.

### BRIEF DESCRIPTION OF THE DRAWINGS

FIG. 1 is a perspective view of a robot-assisted surgical system on which the configurations described herein may be included;

FIG. 2 is a perspective view of a robotic manipulator arm with an instrument assembly mounted to the end effector;

FIG. 3 is a perspective view showing the end effector of the manipulator of FIG. 2, with the surgical instrument mounted to the end effector;

FIG. 4 is a perspective view similar to FIG. 3, showing the surgical instrument separated from the end effector;

FIG. 5 schematically shows a cross-section view of an end effector, taken transverse to the longitudinal axis of the end effector, utilizing an arrangement of detectors to detecting proximity of the end effector to other components or personnel;

FIG. 6 shows a plan view of two end effectors with mounted cameras, and schematically depicts the use of parabolic lenses to increase the fields of views of the cameras;

FIG. 7 is similar to FIG. 6 but shows an embodiment in which infrared LEDs are used to aid in proximity sensing.

FIG. 8 is a block diagram schematically depicting components of an exemplary proximity sensing system;

FIG. 9 schematically illustrates a series of steps for using the system depicted in FIG. 8.

FIG. 10 is a block diagram schematically depicting components of a second exemplary proximity sensing system;

FIG. 11 schematically illustrates a series of steps for using the system depicted in FIG. 9.

### DETAILED DESCRIPTION

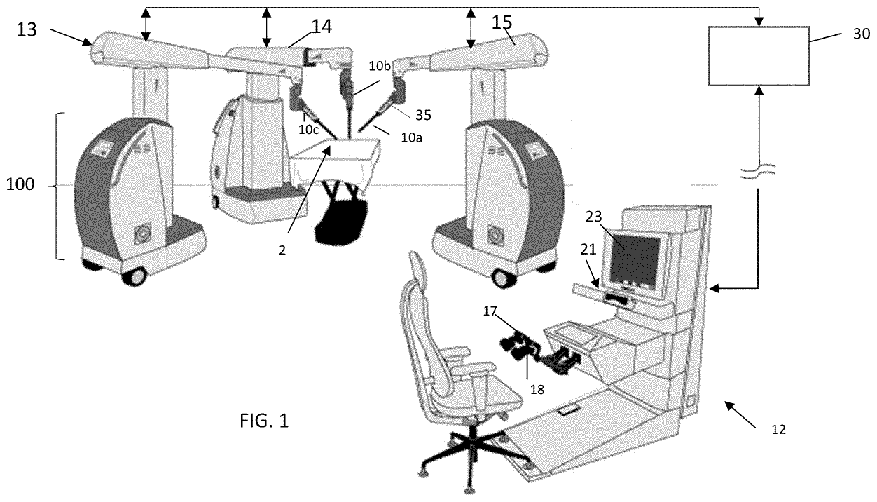

Although the inventions described herein may be used on a variety of robotic surgical systems, the embodiments will be described with reference to a system of the type shown in FIG. 1. In the illustrated system, a surgeon console 12 has two input devices such as handles 17, 18 that the surgeon selectively assigns to two of the robotic manipulators 13, 14, 15, allowing surgeon control of two of the surgical instruments 10a, 10b, and 10c disposed at the working site at any given time. To control a third one of the instruments disposed at the working site, one of the two handles 17, 18 may be operatively disengaged from one of the initial two instruments and then operatively paired with the third instrument. Or, as described below, an alternative form of input such as eye tracker 21 may generate user input for control of the third instrument. A fourth robotic manipulator, not shown in FIG. 1, may support and maneuver an additional instrument.

One of the instruments 10a, 10b, 10c is a laparoscopic camera that captures images for display on a display 23 at the surgeon console 12. The camera may be moved by its corresponding robotic manipulator using input from an eye tracker 21, or using input from one of the input devices 17, 18.

The input devices at the console may be equipped to provide the surgeon with tactile feedback so that the surgeon can feel on the input devices 17, 18 the forces exerted by the instruments on the patient's tissues.

A control unit 30 is operationally connected to the robotic arms and to the user interface. The control unit receives user input from the input devices corresponding to the desired movement of the surgical instruments, and the robotic arms are caused to manipulate the surgical instruments accordingly.

In this embodiment, each arm 13, 14, 15 is separately positionable within the operating room during surgical set up. In other words, the bases of the arms are independently moveable across the floor of the surgical room. The patient bed 2 is likewise separately positionable. This configuration differs from other systems that have multiple manipulator arms on a common base and for which the relative positions of the arms can thus be kinematically determined by the system.

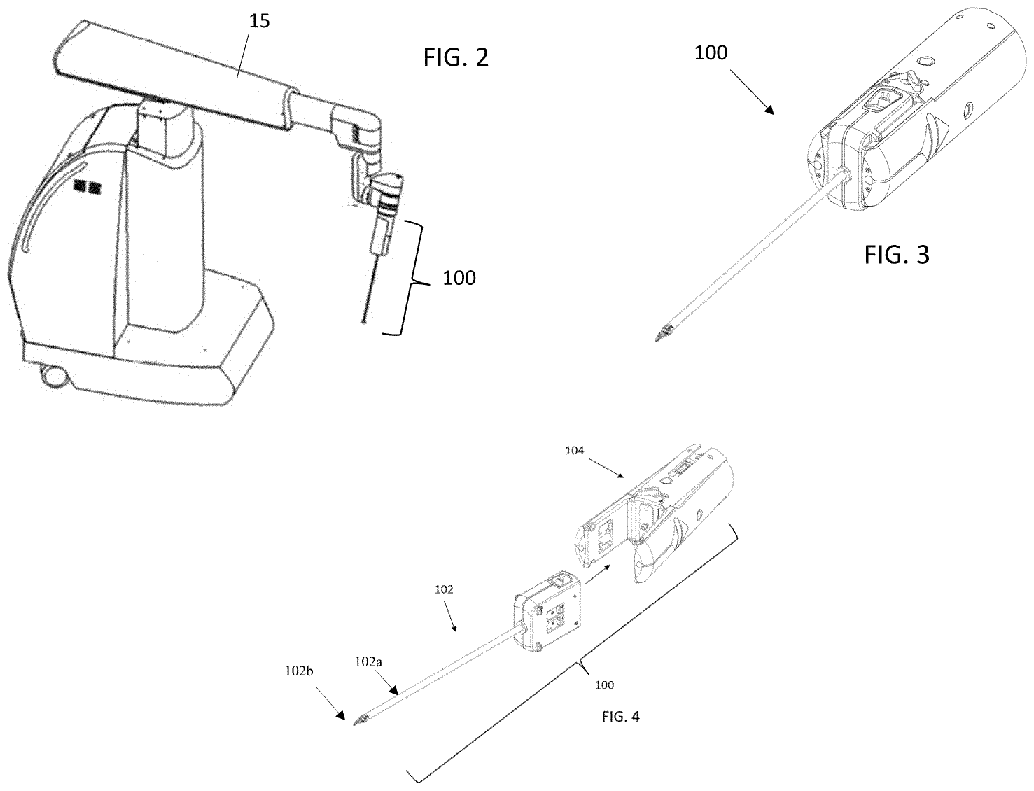

Referring to FIGS. 2-4, at the distal end of each manipulator 15 is an assembly 100 of a surgical instrument 102 and the manipulator's end effector 104. In FIGS. 3 and 4, the end effector 104 is shown separated from the manipulator for clarity, but in preferred embodiments the end effector is an integral component of the manipulator arm. The end effector 104 is configured to removably receive the instrument 102 as illustrated in FIG. 4. During a surgical procedure, the shaft 102a of the surgical instrument is positioned through an incision into a body cavity, so that the operative end 102b of the surgical instrument can be used for therapeutic and/or diagnostic purposes within the body cavity. The robotic manipulator robotically manipulates the instrument 102 in one or more degrees of freedom during the course of a procedure. The movement preferably includes pivoting the instrument shaft 102a relative to the incision site (e.g., instrument pitch and/or yaw motion), and axially rotating the instrument about the longitudinal axis of the shaft. In some systems, this axial rotation of the instrument may be achieved by rotating the end effector 104 relative to the manipulator. Further details of the end effector may be found in commonly owned US Publication 2021/169595 entitled Compact Actuation Configuration and Expandable Instrument Receiver for Robotically Controlled Surgical Instruments, which is incorporated herein by reference. These figures show but one example of an end effector assembly 100 with which the disclosed system and method may be used. It should be understood that the system and method are suitable for use with various types of end effectors.

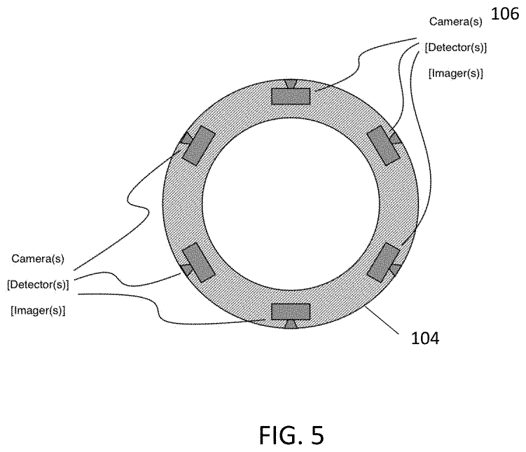

Referring to FIG. 5, a system for predicting collisions may include one or more imagers 106 (also referred to herein as cameras or detectors, etc.) positioned on a portion of a robotic manipulator, such as on the end effector 104. The view shown in FIG. 5 is a cross-section view of the end effector taken transverse to the longitudinal axis of the end effector (which typically will be parallel to the longitudinal axis of the instrument 102). The imagers are depicted as cameras positioned facing outwardly around the perimeter of the end effector as shown. In the drawing, the cameras are shown circumferentially positioned around the circumference of an end effector having a cylindrical cross-section, such that the lenses of the cameras are oriented radially outward from the end effector.

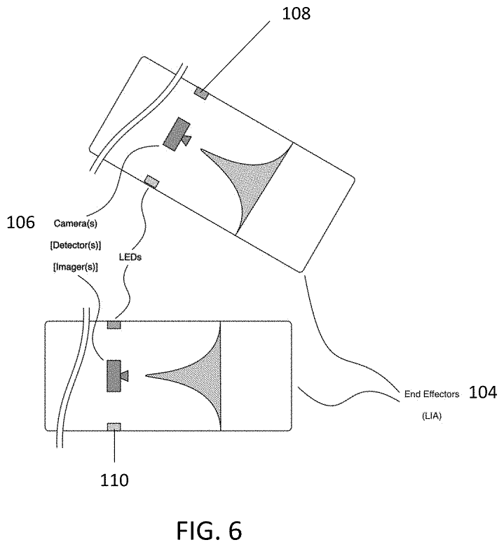

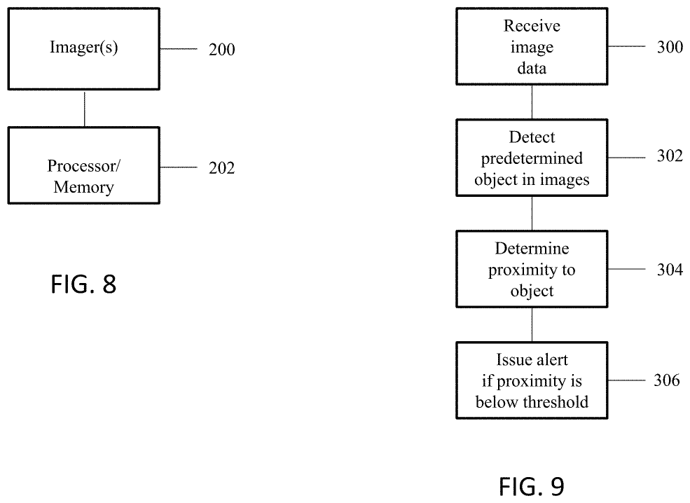

The imager system is used in conjunction with at least one processor, as depicted in the block diagram shown in FIG. 8. The processor has a memory storing a computer program that includes instructions executable by the processor. These instructions, schematically represented in FIG. 9, including instructions to receive the image data corresponding to images captured the imager(s)/camera(s) (300), to execute an algorithm to detect equipment, personnel or other objects in the images (302), and to determine the distance between the manipulator and nearby equipment/personnel (the “proximal object”) or, at minimum, to determine that an object is in proximity to the end effector (304). The proximity detection step may rely on a variety of functions, including, for example, proximity detection, range estimation based on based on motion of feature(s) detected between frames of the captured image data, optical flow, three-dimensional distance determination based on image data from stereo cameras. Where multiple imagers are used, as in FIG. 5, image data from all or a plurality of the imagers may be used in the proximity detection step. In some embodiments, information from multiple cameras may be stitched together to acquire a seamless panoramic view/model that can be used to provide the system with situational awareness view with respect to each degree of freedom of movement of the end effector. In some embodiments, kinematic data from the robotic manipulator may additionally be used to determine proximity, informing the processor where the relevant imagers of the end effector are relative to some fixed point on the corresponding manipulator or some other point in the operating room. Where markers are used on end effectors or other components of a robotic manipulator as discussed with respect to FIG. 6, kinematic data from the manipulator on which the LEDs or other markers are positioned may additionally be used by the proximity detection algorithm and/or by a collision avoidance algorithm.

In some embodiments, the algorithm further determines whether the distance is below a predetermined proximity threshold, and optionally takes an action if the distance is below the predetermined proximity threshold. Exemplary actions include generating an auditory alert or a visual alert (306). A visual alert might result in illumination of a light or LED, or in the display of an alert on a screen or monitor positioned. In either case, the device displaying the alert may be one on the manipulator, at the surgeon console, or elsewhere in the operating room. Other actions might include delivering a haptic alert to one or both of the surgeon controls 17, 18. For example, motors of the surgeon controls may be commanded to cause a vibration that will be felt by the surgeon holding the handles of the controls. Alternatively, the motors may be caused to increase resistance to further movement of the relevant control 17, 18 in a direction that would result in movement of the manipulator closer to the proximal object. Another action, which may be in addition to the alert 206 or an alternative to the alert 306, may be to terminate motion of the manipulator, or to terminate or slow-down motion of the manipulator that would result in movement of the manipulator closer to the proximal object. Similar actions may be taken in a simpler configuration where the sensitivity of the imagers/detectors is such that the system simply determines that there is an object in proximity to the end effector.

More complex actions may include providing updated motion to the manipulator or setup linkages with redundant kinematics to gradually move joints to minimize the likelihood of collisions between specific portions of the manipulator or to move the entire manipulator to overall configurations that are less likely to collide. This configuration optimization would occur in a mode that is largely transparent to the user or could be a mode that the user enables when it is determined to be safe to do so. Safe contexts for use of the feature might include times when there are no surgical assistants working near the manipulator, when the instruments are in the trocars or not yet installed on the end effector.

In some implementations, the collision prediction/detection algorithms are processed for a single arm only on its own processing unit. In other implementations, they are processed in a single, central processing unit that collects information from a variety of inputs/manipulators/systems and then provides input commands to arms or other system components.

In a modified embodiment, imagers on the end effector might include one or more camera(s) having a parabolic lens, an axisymmetric lens or a reflector. Such lenses and reflectors allow a single lens to cover a very wide field of view. In configurations using them, the processor 202 is further programmed to mathematical unwarp the images captured by the image data into an appropriate spatial relationship. Some implementations may be configured to additionally permit forward viewing using the imager, such as by providing a gap or window in the parabolic lens, asymmetric lens or reflector. The shape(s) of the reflectors chosen for this embodiment may be selected to allow for targeting viewing of regions of interest, such as regions where problematic proximal objects are most likely to be found. Other implementations may use two cameras, one to cover each hemisphere and allow for use of the central axis of the structure for other purposes.

In alternative embodiments, omni-directional cameras may be used for sensing proximity between end effectors or other components. One or more such omni-directional cameras may be positioned on the end effector, elsewhere on the manipulator arm (e.g., high on the vertical column of the arm shown in FIG. 2, or on the horizontally extending boom), or at a high point in the operating room, such as on a ceiling fixture, cart, laparoscopic tower, etc.

As shown in FIG. 6, end effectors (or other potential proximal objects) in any of the disclosed embodiments may include known features, patterns, fiducials, LEDs that may be detected in the image data captured by the cameras, and used for predicting potential collisions. The LEDs may vary in color depending on their position on the end effector, allowing the system to determine through image analysis which end effector or other proximal object is being captured by the relevant imagers. For example, for each end effector shown in FIG. 6, a green LED 110 is positioned on the right side of the end effector and a red LED 108 is positioned on the left side.

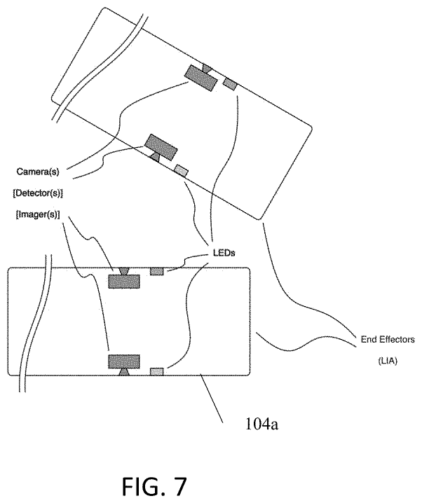

Infrared (IR) LEDs may be used in some embodiments for tracking and collision detection, as illustrated in FIG. 7. For example, LEDs that emit infrared wavelengths of light may be installed on the end effector or other elements of the robotic surgical system. Infrared light may transmit through sterile drape material so that when the end effector is covered by a sterile drape for surgery, the infrared light will transmit through it and can thus be detected by the imagers of the other end effectors. In some embodiments, the IR LEDs may be positioned beneath the housing/skin 104a (FIG. 7) enclosing the internal components of the end effector, since the IR light can transmit through visibly opaque materials. These LEDs may be single, or may be arranged in a certain pattern, and/or may use flash/blink patterns to provide different information, or to differentiate between elements and/or sides of a robot part. These LEDs or patterns of LEDs may be detected with an optical detector or a camera. While IR LEDs may be preferable, LEDs that emit in alternate or additional wavelengths (visible or invisible, RGB, etc.) are within the scope of the invention. Techniques described co-pending application Ser. No. 17/944,170 may be used to determine the distances from the optical detector or camera to the tracked component.

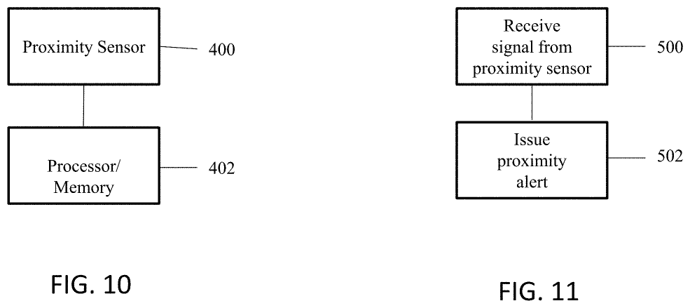

Referring to FIGS. 10 and 11, alternative types of proximity sensors such as capacitive sensors or inductive sensors may be used as an alternative or in addition to the optical detectors described above. For example, a capacitive element or series of elements may be monitored by a system to detect proximity to another capacitive element, series of elements, or other objects that may have a capacitive effect—such as a part of a user or patient's body. In addition, these capacitive elements may be used to detect contact/collisions, whether as a primary source or a secondary/backup sensor. As yet another example, an inductive proximity sensor may be used to detect proximity between metallic components of the surgical system, such as end effectors or other portions of the manipulator. These alternative proximity sensors may be individual sensors, or a plurality of sensors placed in multiple positions on the end effector, such as in a circumferential arrangement as described with respect to the imagers shown in FIGS. 5-7.

It should be mentioned that while these embodiments are described with respect to the end effector of a manipulator, the same principles may be used to obtain overall situational awareness in the OR, potentially with a similar camera/lens/reflector configuration mounted on another portion of a manipulator arm, the vertical axis of the manipulator arm, etc.

All patents and applications referred to herein, including for purposes of priority, are incorporated herein by reference.

What is claimed is:

1. A robotic surgical system comprising:
a first robotic manipulator arm having a first base and a first end effector configured to support a first surgical instrument;
a second robotic manipulator arm having a second base and a second end effector configured to support a second surgical instrument;
each of the first base and the second base independently moveable on a floor of an operating room;
proximity sensors positioned on at least one of the first end effector and the second end effector to detect proximity of the first end effector to the second end effector.

2. The system of claim 1, wherein the proximity sensors comprise imagers on said at least one of the first end effector and the second end effector.

3. The system of claim 3, wherein the images comprise a plurality of imagers circumferentially positioned around the end effector.

4. The system of claim 2, wherein the imagers are positioned on the first end effector and wherein the system further includes a plurality of light emitters on the second end effector.

5. The system of claim 4, wherein the light emitters are circumferentially positioned on the second end effector.

6. The system of claim 2, wherein the imagers are positioned on the first end effector and the second end effector, wherein the system further includes a plurality of light emitters on each of the first end effector and the second end effector.

7. The system of claim 1, wherein the proximity sensor comprises
a camera positioned on at least one of the first end effector and the second end effector, the camera including a parabolic lens.

8. The system of claim 7, wherein the camera is an omni-directional camera.

9. The surgical system of claim 1, wherein the proximity sensor is a capacitive sensor on at least one of the first and second manipulators, the capacitive sensor configured to detect when the first end effector is in proximity to the second end effector.

10. The surgical system of claim 1, wherein the proximity sensor is
an inductive sensor on at least one of the first and second manipulators, the inductive sensor configured to detect when the first end effector is in proximity to the second end effector.
